(19) United States
(12) Patent Application Publication
(10) Pub. No.: US 2024/0302271 A1
(43) Pub. Date: Sep. 12, 2024

(54) Portable, rechargeable soil respiration and carbon dioxide sensor drill

(71) Applicant: Sabrina Lenae Williams, Los Angeles, CA (US)

(72) Inventor: Sabrina Lenae Williams, Los Angeles, CA (US)

(21) Appl. No.: 18/119,668

(22) Filed: Mar. 9, 2023

(51) Int. Cl. G01N 21/3504; G01N 1/08; G01N 33/24

(52) U.S. Cl. CPC G01N 21/3504; G01N 1/08; G01N 33/24

(57) ABSTRACT

A portable, rechargeable device for measuring concentrations of soil gases in situ through drill function. The device comprises a singular body enclosing a hardware interface connected to a processing unit, a gas sensor in a permeable chamber, a data module to interact with the sensor, a drill function, a display to report data collection results, a wireless communication module and GPS. A method for measuring soil gas comprising insertion of the portable device into soil through a drill function and diffusion of gases into a hollow cylindrical body through a gas-permeable fabric facilitating contact with the sensor by passive gas permeation. A system for connecting the device to a network to store the data in a database, for calibration and comparison of sensor data to existing data.

### CROSS REFERENCE TO RELATED APPLICATIONS

This application claims the benefit of U.S. Provisional Application Ser. No. 63/317,976, filed by Sabrina Williams on Mar. 9, 2022, entitled “Portable, rechargeable soil respiration and carbon dioxide sensor drill,” commonly assigned with this application and incorporated herein by reference.

### STATEMENT RE: FEDERALLY SPONSORED RESEARCH/DEVELOPMENT

Not Applicable.

### BACKGROUND OF THE INVENTION

### Field of the Invention

The invention relates generally to an apparatus, system and method for measuring concentrations of soil gases in situ. Specifically, this invention relates to a portable apparatus to determine and report concentrations of soil respiration and carbon dioxide and estimations of soil organic carbon in real time using nondispersive infrared spectrometry.

Carbon dioxide (CO₂) in the atmosphere is overheating Earth's biosphere and producing global warming and pollution. One natural and efficient way of removing excess greenhouse gases is to sequester CO₂ in agricultural soils. Carbon sequestration is the process of capturing and storing atmospheric CO₂ and is primarily facilitated through photosynthesis in plants through plant respiration, soil surface CO₂ efflux achieved through emission of CO₂ by microbial respiration—becoming the largest flux in the terrestrial carbon cycle. The carbon that remains in the plant tissue transforms into organic matter through a series of chemical reactions caused by decomposing microbes. Carbon sequestration has the dual benefit of reducing CO2 levels in the air while improving soil health which leads to higher yields for farmers and other growers.

To encourage carbon sequestration, programs have been developed to compensate farmers for the tonnage of CO₂ they remove from the atmosphere by converting it into soil organic matter (SOM) which contains soil organic carbon (SOC). Unfortunately, carbon is notoriously unstable—and measuring exact carbon pools in soil has been historically difficult due to complex carbon dynamics. Generally it must be measured in a laboratory setting using loss of ignition (LOI) method, where oven dried samples are heated up to 400° C. and organic carbon is calculated based on percent weight loss during the ignition step.

It would be desirable, therefore, if an improved method and apparatus was available which facilitated the calculation and reporting of SOC content in soil and variations in SOC content in soil over time. Such an improved method and apparatus might deliver benefits of sensor technology to farmers and growers who have been denied those benefits, due to cost or unwieldiness. These farmers and growers include, by some estimates, over a half-billion people farming on smallholder sites, residential plots, community gardens, urban farms and other small acreage—growing food and plants in some of the hottest and most polluted urban and peri-urban environments. When their soil is unhealthy, many smallholders do not know to what extent, and it is hard to determine without expensive machinery. Therefore, smallholders are challenged to take steps to remediate the soil to sequester carbon and keep it out of the atmosphere, which could lead to less pollution in their communities.

Various gases, such as CO₂, absorb narrow bands of energy in the infrared region of the optical spectrum. A non-dispersive infrared, or NDIR, sensor makes use of this property to measure the amount of the gas in question in the ambient atmosphere by emitting light from an infrared source and using a photosensor to measure the amount of energy absorbed by the gas in question. Most often, this process has made NDIR only suitable for measuring CO₂ in ambient air through continuous environmental monitoring (as in household or greenhouse structure detectors).

NDIR spectroscopy is useful in agricultural settings because it can provide real-time monitoring of soil respiration and collected actively into small chambers. Chamber-based measurements allow direct measurement of CO₂ efflux from soils on a small scale, and spectroscopy offers a non-destructive alternative to conventional SOC testing. NDIR sensors have been used in discrete sampling through placement of sensors in top-of-soil, dynamic, open-or-closed chambers (devices that directly measure emissions from the soil with or without introducing ambient air), which are immobile. In some methods of measuring soil gas, multiple, individual NDIR sensors are placed at multiple locations of a site to measure soil respiration, risking loss or other disturbance of the sensors once the user moves to other parts of the site. NDIR sensors are also used in manual soil probes in which multiple gases (carrier gas and soil gas) are combined in complex action (via inlet and outlet mechanisms) to produce a result for analyzing soil temperature.

Although extremely inexpensive, NDIR is not prominent in commercial applications of soil carbon measurement, because of prominence of (and bias for) measurements analyzed ex situ in closed laboratory systems, which larger, commercial agriculture enterprises find more convenient and can afford. The commercially available soil respiration, SOC and CO₂ sensors and measurement tools that use infrared sensors in situ are of large form and for large-scale projects. When these large form devices are deployed, there is limited portability and placing them at proper soil depths up to 30 cm requires skill most farmers, hobby gardeners and other smallholders lack.

Smallholders may use a probe with integrated NDIR to reach below-surface of soil to take measurements, but the method can fail to collect sufficient or consistent information because of limited circumference of the probe and limited depths reached, since users have varying strength to effectively strike the probe. The user is also expected to accurately and consistently count elapsed time of collection of soil gas before recording or logging the information, even when not skilled in using such probes. Inexpert methods fail to get the right depth or timing.

A key ambition in soil health and carbon reporting is to integrate enough ground-truthed soil measurements (those using in situ observations to confirm accuracy of remotely sensed data) to feed strong data models. Measurements from in situ NDIR spectroscopy can serve as reliable inputs to data models that also rely on GPS and satellite derived soil databases to estimate carbon sequestration. There is no current existing device or method that simultaneously measures CO₂ and SOC gases at proper soil depth, compares those gases to soil type from known soil database sources, and reports data in real-or near-real-time, using a portable, low-cost apparatus that is both chamber and probe.

### BRIEF SUMMARY OF THE INVENTION

The invention is an apparatus, method and system related to the in situ measurement and reporting of soil gas efflux, namely, soil respiration, carbon dioxide (CO₂) and estimations of soil organic carbon (SOC). Said apparatus comprises a hollow cylindrical body defining a chamber, means for receiving said gases within said chamber, a microcontroller unit (MCU), means for capturing and reporting data related to sensor measurement, and a motorized electric drill, means for the chamber to enter the soil to collect said gases, namely resulting from soil respiration, CO₂ content of soil and estimations of SOC. The method of the invention uses non-dispersive infrared (NDIR) sensing technique or principle to determine soil gas efflux, namely soil respiration, CO₂ content of soil and estimations of SOC. The invention provides a system for determining the soil gas efflux, namely soil respiration, CO₂ content of soil and estimations of SOC and/or variations of the soil gas efflux, namely soil respiration, CO₂ content of soil and estimations of SOC.

The apparatus of the invention includes a motorized electric drill and NDIR sensor including an ergonomic handle, which may move longitudinally within a hollow cylindrical body. In some embodiments the ergonomic handle may be teardrop shaped. In other embodiments the ergonomic handle may be an angled element of the hollow cylindrical body. The apparatus includes a light-emitting diode display (LED), or other means of displaying data about operation of the apparatus, including but not limited to battery levels, elapsed time, sensor data, power, and other instruction or information that may permit use of the invention, and which LED, in some embodiments, is housed in the ergonomic handle.

The apparatus includes a motor connected and configured to drive a separate, permeable chamber (of porous material or other means of permitting gases through its surface) to rotate within the hollow cylindrical body, a rechargeable battery configured to power the motor, and an MCU configured to control the motor and electrically connected to the motor and the rechargeable battery, all of which in some embodiments may be housed in the ergonomic handle. Further, the rechargeable battery is preferably a lithium battery, which is housed in or proximal to the ergonomic handle. Further, the apparatus may generally be provided with a power interface and may connect to an external power supply. The power interface is electrically connected to the MCU to power the motor and charge the rechargeable battery. Preferably, the ergonomic handle supports or has embedded the MCU and a wireless technology that connects to electrical elements in or proximal to a circular disc that holds or caps an NDIR sensor, where in some embodiments, the wireless technology used may be Bluetooth, Wi-Fi, ZigBee, low-power wide-area (LPWA) network or long range (LoRa) wireless communication. Further, the ergonomic handle is provided with a control button, which may be called a trigger, which is electrically connected to the controller, and the control button controls on/off and forward/reverse rotation of the motor.

Preferably, the motor is connected within the ergonomic handle with clearance to rotate freely at or proximal to the base of the handle. Preferably, the motor is connected, substantially coaxially, at its bottom to a circular disc that holds or caps an NDIR sensor. Preferably, said circular disc supports or is connected to or has, dedicated to the NDIR, an embedded microcontroller or microcontroller board and a wireless technology, which connects to electrical elements in or proximal to the handle.

Further, the circumference of the motor is of a size that permits movement and entry through the hollow cylindrical body, which in some embodiments has an annular cap at one end to receive the motor. Preferably, the circumference of the circular disc at the bottom of the motor is of a size to prevent displacement of the disc through the annular cap of the outer hollow cylindrical body. Preferably, the circular disc is attached in permanent fashion to an elongated permeable chamber that may replicate the function of a shaft or spindle. Further the permeable chamber that may replicate a shaft or spindle may enclose an NDIR sensor. Further the permeable chamber is defined with a bottom surface that may replicate the function of a drill chuck. Preferably the drill bit connects, substantially coaxially, to the surface that replicates the function of a drill chuck, to exit from the hollow cylindrical body.

The hollow cylindrical body is of elongated form and has a central longitudinal axis with an inlet for introducing soil gas at one end and closed by an ergonomic handle at the other end. Preferably the hollow cylindrical body is of a tubular configuration and thus is of a circular cross section. Further the hollow cylindrical body may be encircled by and connected near its top to an annular cap. The hollow cylindrical body may be located substantially coaxially to the motor. The clearance between the hollow cylindrical body and motor, circular disc, NDIR sensor and permeable chamber, allows longitudinal movement of the motor, circular disc, NDIR sensor and permeable chamber, within the hollow cylindrical body. The hollow cylindrical body may include means adapted for cooperation with the motor, circular disc, NDIR sensor and cylinder, to limit movement of the motor, circular disc, NDIR sensor and permeable chamber.

The current embodiment of the invention includes a method for the apparatus to enter the soil to collect gases, namely resulting from soil respiration, CO₂ content of soil, to calibrate to estimations of SOC.

Preferably gases are passively realized through permeation at the bottom of the hollow cylindrical body. The means of gas permeation may comprise a grid, grating or screen. The grid, grating or screen suitably is in a similar configuration to, and size of the cross section of the hollow cylindrical body. The grid, grating or screen is annular in shape to accommodate a drill bit and may include a ring, which may be a washer, through which the drill bit moves. The ring may be of a durable, rubber material, suitable for sealing and damping applications, which permits the drill bit to freely move. The grid, grating or screen may include an outer ring capable of twisting or screwing engagement with the hollow cylindrical body. A drill bit in the current embodiment may be a Forstner type or one that removes soil elements away from the apparatus. The circumference of the shank of the drill bit is of a size that permits movement through an opening of the annular grid, grate or screen.

In some embodiments, a gas-permeable material constituting a fibrous filter or pad may be supported on the grid, grating or screen, to occlude entrance of fine particles of soil into the hollow cylindrical body through the grid, grating or screen. In one embodiment of the invention, the fibrous filter is a hydrophobic, gas-permeable material. In another embodiment, the gas-permeable material or pad may comprise an air permeable pliable fabric. Preferably the gas-permeable material forms or adopts an identical configuration upon the grid, grating or screen. Preferably the gas-permeable material forms a substantial seal with the side wall of the hollow cylindrical body to prevent or minimize soil entering the chamber.

The current embodiment of the invention includes a system for reporting longitude, latitude and altitude measurement indicating a point to be detected, wherein the apparatus acquires geographical coordinates of the point to be detected from the Global Positioning System (GPS), and a computer automatically calibrates a soil type or profile from a comparative database of soil type or profile corresponding to the point to be detected. In some embodiments of the invention, the soil type or profile database is a global map of soil type or profile details derived from GPS or satellite. In another embodiment of the invention, the point to be detected corresponds to a soil type or profile database to calibrate estimates of SOC. In some embodiments of the invention, the system generates a visual image with Geographic Information System (GIS) visualization system, of the location and soil respiration or CO₂ data acquired from the apparatus calibrated to a soil type or profile or estimates of SOC. In an embodiment of the invention, the estimate of SOC and distribution of SOC is derived from publicly available geographical soil datasets. In some embodiments of the invention, the location and soil respiration or CO₂ data acquired from the apparatus is calibrated to data modeled on the carbon content in the surface horizon of soil derived from digital soil mapping (DSM).

In some embodiments of the invention, the instructions for processing location, NDIR sensor data are stored in the MCU and later (or potentially in real time) transmitted via wireless communication to a smart phone, tablet, computer or other PDA. The present embodiment of the invention encompasses wireless communication but wired communication may be desirable in some applications and can be accommodated by this invention. Some embodiments of the invention may include an application or computer program embodied on a non-transitory memory device that stores collected data. Moreover, as used herein, the term “non-transitory memory device” includes all tangible, computer-readable media, including, without limitation, non-transitory computer storage devices, including without limitation, volatile and non-volatile media, and removable and non-removable media such as firmware, physical and virtual storage, CD-ROMS, DVDs, and any other digital source such as a network or the Internet, as well as yet to be developed digital means, with the sole exception being transitory, propagating signal.

In some embodiments of the invention, data collected by the apparatus may be stored in memory off-line and/or transmitted and/or synchronized with a database system and/or a cloud memory, wherein synchronization can be automatic, as soon as a network connection is available and/or initiated manually or the data may be synchronized in real-time and/or, in particular temporarily, stored off-line, preferably for later synchronization.

The present invention introduces a handheld, economical carbon sensor for in-situ use that detects CO₂ concentration in a soil profile, and provides real-time, basic data in calculations of the global carbon in the soil profile. Because soil carbon is so labile, one approach is to directly measure factors, or proxies, of soil biology that reflect the presence of carbon. At least two of these factors, or proxies, are measurable by the current embodiment using NDIR sensoring: active carbon and respiration. Knowledge about soil carbon levels assists with the sequestration activities that result in reductions in greenhouse gas emissions.

Rapid, site-specific soil properties analyses are imperative for monitoring agricultural soil conditions that inform management practices related to soil carbon monitoring, reporting and sequestration. The present invention inexpensively and effectively measures soil carbon at the field scale. In this recent patent disclosure, the present inventor takes advantage of the fact that an apparatus for measuring soil carbon in situ with NDIR spectroscopy, with a lightweight, singular, self-contained body and data-acquisition system is an innovative approach that achieves a concomitant outcome of broad consumer access to such an apparatus.

Those skilled in the art will appreciate that the concept of this disclosure may be readily utilized as a basis for the design of other structures, methods, and systems for carrying out the several purposes of the rechargeable tool having NDIR CO2 sensor and wireless signal collection capabilities.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is an exterior elevation view of an apparatus in accordance with an embodiment of the present invention.

FIG. 2 is an exterior elevation view of an apparatus in accordance with an embodiment of the present invention highlighting the position after drilling into soil.

FIG. 3 is a cross-sectional view of an embodiment of the present invention configured for receiving soil gases.

FIG. 4 is a cross-sectional view of the upper hollow cylindrical body of the embodiment of FIG. 3 showing a wireless coil, circumference of a handle and drill bit.

FIG. 5 is a cross-sectional view of the lower hollow cylindrical body of the embodiment of FIG. 3 highlighting a permeable grid, grating or screen.

FIG. 6 is a block diagram illustrating components of a system for receiving, transmitting and storing sensor data.

While the invention is susceptible of various modifications and alternative constructions, a certain illustrative embodiment thereof has been shown in the drawings and will be described below in detail. It should be understood, however, that there is no intention to limit the invention to the specific forms disclosed, but on the contrary, the intention is to cover all modifications, alternative constructions and equivalents falling within the spirit and scope of the invention as defined by the appended claims.

### DETAILED DESCRIPTION OF THE INVENTION

The following detailed description is merely exemplary in nature and is not intended to limit the described embodiments of the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims. Furthermore, there is no intention to be bound by any expressed or implied theory presented in the preceding technical field, background, brief summary or the following detailed description.

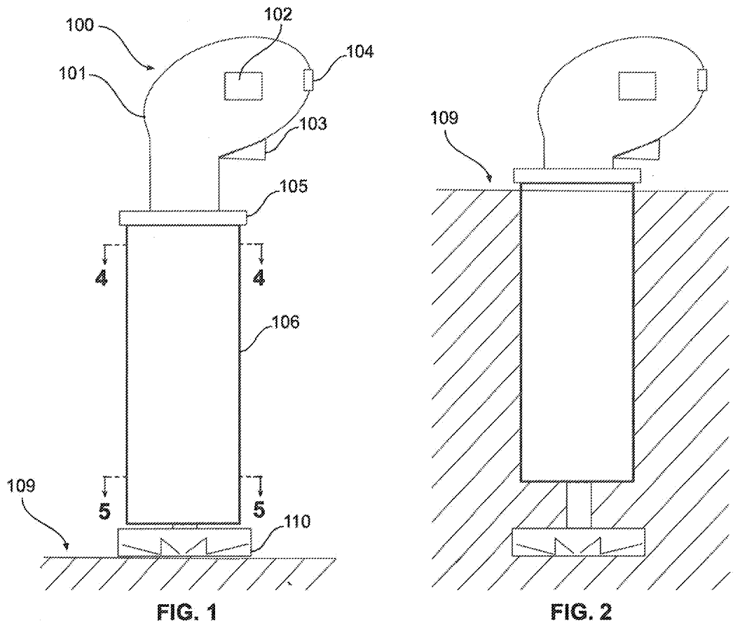

As best illustrated in FIGS. 1 and 2, the soil carbon sensor (hereinafter the apparatus) (100) generally comprises a handle (101), and a hollow cylindrical body (106) defining a chamber of elongated form.

Referring to the drawings and firstly to FIG. 1, the invention collects a sample or samples of soil gas efflux, namely organic carbon, soil respiration and carbon dioxide for the purpose of determining the amount or content thereof. Power to the apparatus is triggered via switch (103). The electronics activated and displayed by an LED (102) include a light to indicate on/off status, a screen to read the on/off status and/or initial reading of the sensor, and/or the power level of the rechargeable battery, and/or any other information about use of the apparatus. There may be a period of time before the apparatus provides a reading of gas measurement. As illustrated in FIG. 1, the apparatus may then be set at its drill bit end on the surface of a natural ground (109) to initiate entering that surface. The apparatus can be used to measure certain gases, especially carbon dioxide, and soil respiration below the surface of a natural ground (109). The apparatus may be about 6 cm diameter and 32 cm long in an undeployed state. These dimensions are demonstrative and not exhaustive, specific or final.

Regarding FIG. 1, the invention handle (101) is of hollowed construction. The handle (101) is curved in shape and adapted to be grasped via a user, for pointing downward. The handle (101) is further defined with, at minimum, a compartment for a rechargeable battery (314), LED display (102), and a motor (301) with pin that is in wired connection with a trigger (103). The trigger, which may or may not integrate with a forward/reverse lever or button, is in wired connection with the rechargeable battery (314). In order to access electricity that is used to recharge the rechargeable battery encased within the handle, the apparatus may be connected to an external power source (104).

FIG. 2 shows the introduction of the apparatus into a natural ground from the surface (109).

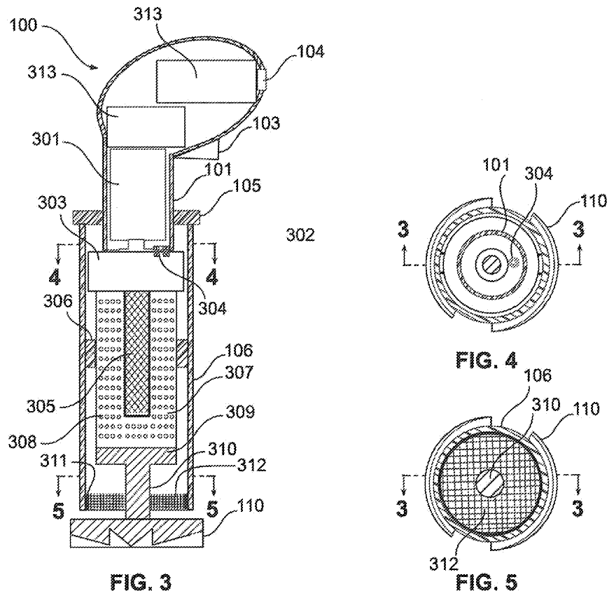

As illustrated in FIG. 3 and FIGS. 4-5, the handle (101) encloses at least a microcontroller unit (MCU) (313), a motor (301), an LED display (102) and rechargeable battery (314), wherefrom electronics of the apparatus may connect to the power source. The bottom of the motor includes or attaches to a circular disc (303). The circular disc (303), which may also be called NDIR sensor housing, is of appropriate height to house the elements to operate an NDIR sensor, including but not limited to a dedicated microcontroller or microcontroller board (MCB), Bluetooth node or similar wireless communications element and other electronics of an NDIR sensor (305), which in some embodiments. The MCB with Bluetooth or similar element integration housed in the circular disc (313) connects to the NDIR sensor. In some embodiments, the NDIR sensor (305) is connected directly and only to the MCU. The circular disc (303) is attached to a permeable chamber (307) that surrounds the NDIR sensor (305). Electrical power may flow between the circular disc (303) and the MCB and Bluetooth or similar element via contact with a wireless coil assembly (304), for purposes of powering and recharging the NDIR sensor (305).

The permeable chamber is an elongated housing (307), which is of a circular cross section so as to be of a tubular configuration. The permeable chamber is of a porous material or may be permeable or may have other means of permitting gases through its surface (308) as a collection chamber. The permeable chamber has a central longitudinal axis attached at top to a circular disc (303) and attached at bottom to an element which may replicate the function of a drill chuck (309).

The drill chuck element (309) may attach to a drill bit (311) which may be of Forstner type, the shank of which (310) moves through the center of a grid, grating or screen (312). The drill bit (311) is of a diameter that permits the hollow cylindrical body to move into soil. The motor (301) may be in mechanical connection with the drill chuck element (309).

The apparatus includes a permeable grid, grating or screen (312) through which gases may passively enter the hollow cylindrical body (106). The hollow cylindrical body (106) enables holding of chemical gas for an appropriate amount of time to permit passive transfer to the permeable chamber (207). A method for determining the amount or content of soil gas efflux, namely organic carbon, soil respiration and carbon dioxide, includes passive gas permeation to contact the NDIR sensor (305).

The annular grid, grating or screen (312) is connected to the hollow cylindrical body (106) at the outer circumference of the grid, grating or screen. It includes a central opening (312), which can neatly but firmly receive the shank (310) of a drill bit (110) and substantially seal there against during longitudinal movement of the drill bit. In some embodiments, the grid, grating or screen (312) may be bonded or built together. In another embodiment the grid, grating or screen may be screwed or rubberized to adhere. To prevent entry of fine soil particles, the grid, grating or screen may be comprised of a flexible, flat, disc-shaped element formed of a fabric of other pliable material, which is air or gas permeable. The fabric or other pliable material preferably comprises an air or gas permeable material, which can handle moisture encountered in the implementation of the apparatus. A suitable material may comprise a woven fiberglass cloth. The grid, grating or screen may include an outer ring (311) capable of twisting or screwing engagement with the housing or chamber.

The hollow cylindrical body (106) is generally planar, comprised of an elongated tubular housing which is of a circular cross section and which has an annular flange at the top (105). The annular flange permits the longitudinal movement of the handle (101) and motor (301) through its center (105). The annular flange is of wider diameter than the hollow cylindrical body (106) to assist in restricting movement of the handle into the natural ground (109). At an appropriate length on the interior surface of the hollow cylindrical body is affixed an annular element (306) which is of a circumference that restricts longitudinal movement of the circular disc (303).

The apparatus may be formed of various suitable materials. In one implementation, it is formed of a substantially rigid, water-insoluble, fluid-impervious, material where “substantially rigid” is used to mean the material will resist deformation under medium impact with soil.

With respect to the above description, it is to be realized that the optimum dimensional relationship for the various components of the apparatus (100), to include variations in size, materials, shape, form, function, and the manner of operation, assembly and use, are deemed readily apparent and obvious to one skilled in the art, and all equivalent relationships to those illustrated in the drawings and described in the specification are intended to be encompassed by the invention.

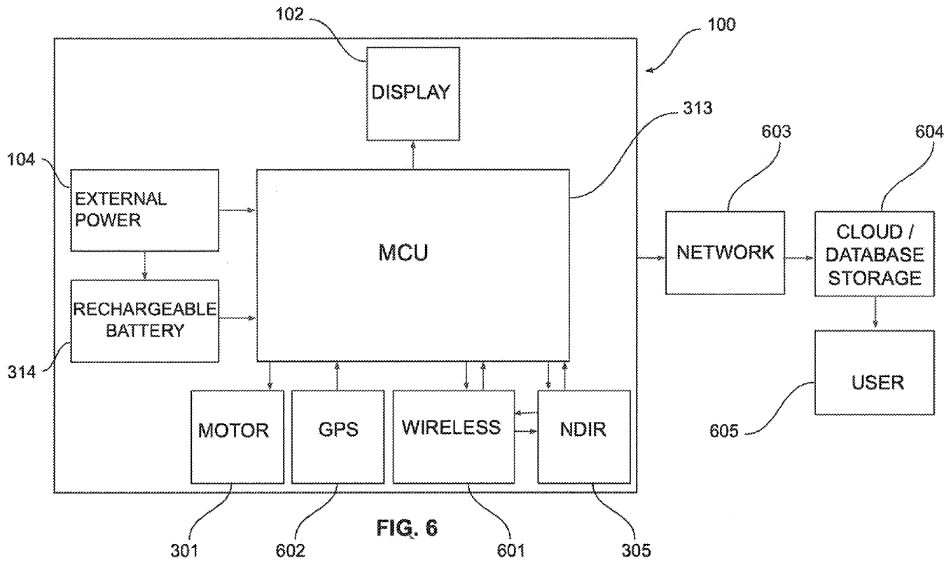

FIG. 6 illustrates the schematics of an operational system of the apparatus that facilitate input to data models that rely on soil databases. The MCU (313) controls interactions of the components of the apparatus, including the LED display (102), the motor (301), Bluetooth (or other wireless communication) node (601). External power (104) may provide power directly to the MCU (313) or to the rechargeable battery (314). The rechargeable battery may provide power directly to the MCU (313). The MCU receives location coordinates from a Global Positioning System (GPS) module (602). The NDIR sensor may be wired to communicate with the MCU (313) directly or via Bluetooth (or other wireless communication) node (601). The apparatus may emit a wireless signal for communication with a network (603), of which network is accessed by cloud storage and databases (604) and then accessible to the user (605).

It shall be noted that those skilled in the art will readily recognize numerous adaptations and modifications which can be made to the various embodiments of the present invention which will result in an improved invention, yet all of which will fall within the spirit and scope of the present invention as defined in the following claims. Accordingly, the invention is to be limited only by the scope of the following claims and their equivalents.

The claims offered hereto in no way limit the inventive matter specified herein; rather, the following claims are intended to be exemplary only, to clarify and illustrate certain embodiments of the present invention.

1. An apparatus for sampling soil gas in situ comprising:
a hollow cylindrical body which hollow cylindrical body having an inlet for introducing soil gas to an NDIR sensor on one end and closed by an ergonomic handle on the other;
said hollow cylindrical body also substantially enclosing a permeable chamber connected to a drill bit;
said permeable chamber having an inlet for introducing soil gas to an NDIR sensor for measuring a concentration of a component of soil gas;
wherein when said apparatus is positioned in a soil gas measurement site, soil gas diffuses into said hollow cylindrical body and can flow to said gas sensor.

2. The apparatus as defined in claim 1 wherein said hollow cylindrical body is elongate and said inlet and drill bit are located at the open end thereof.

3. The apparatus defined in claim 2 wherein said inlet is covered by a membrane being a barrier to liquid, permeable to the soil gas and, such that when said apparatus is positioned in a soil gas measurement site, soil gas diffuses into said hollow cylindrical body, and said membrane having an opening through which said drill bit moves longitudinally.

4. The apparatus as defined in claim 3 wherein the inlet is of fibrous filter occlusive to soil debris, configured to filter particulate matter, protecting the interior of said hollow cylindrical body from said matter in the soil.

5. The apparatus as defined in claim 3 wherein said hollow cylindrical body forms an outer wall substantially enclosing a separate, permeable chamber, wherein said permeable chamber is cylindrical and closed at one end by an impermeable cap attached to the drill bit.

6. The apparatus as defined in claim 5 wherein the NDIR sensor is mounted inside of said permeable chamber at one end.

7. The apparatus as defined in claim 6 wherein said permeable chamber is closed at one end by housing of said NDIR sensor.

8. The apparatus as defined in claim 7 further comprising a housing for the NDIR sensor such that said housing is positioned to introduce said NDIR sensor on one end to soil gas in said permeable chamber and on another end connect to electronic elements and positioned to prevent diffusion of soil gas from said cavity.

9. The apparatus defined in claim 8 wherein said housing encloses said electronic elements for controlling said NDIR sensor and a datalogger for logging data.

10. The apparatus defined in claim 9 wherein said NDIR sensor employs sensing using, a solid-state infrared detector, CMOS compatible (microelectromechanical systems) MEMS, broadband IR detector or MEMS ScAIN-based pyroelectric detectors.

11. The apparatus defined in claim 10 further comprising a motor accommodated in the hollow cylindrical body, connected to a rechargeable battery, to rotationally move said permeable chamber connected to the drill in a vertical direction.

12. The apparatus defined in claim 11 wherein said rechargeable battery supplies power to a microcontroller unit (MCU), and said motor and electric-dependent elements of the apparatus, through a trigger on the apparatus that initiates said power on, off, and forward and reverse motion.

13. The apparatus defined in claim 1 further comprising an LED display at one end of the apparatus, connected to said MCU and said rechargeable battery.

14. A method for taking measurements of a soil gas in situ comprising:
positioning an apparatus, with a hollow cylindrical body and permeable chamber, below the surface of a soil site, allowing a soil gas to contact an NDIR sensor through a diffusion of said soil gas, measuring and recording data of a concentration of said soil gas diffused within said permeable chamber, and logging said data in a system.

15. A method as defined in claim 14 wherein said apparatus is positioned below the surface of said soil site by supplying power to the apparatus to cause an attached drill bit to rotate from the surface to below the surface of said soil site.

16. A system for measuring soil gas comprised of:
An apparatus for sampling soil gas in situ having:
a hollow cylindrical body with ergonomic handle, having an inlet for introducing soil gas to an NDIR sensor and connected to a drill bit;
a permeable chamber for introducing soil gas to an NDIR sensor for measuring a concentration of a component of soil gas;
an MCU for controlling said apparatus and logging said soil gas data;
communication with a remote computing system via a wireless communications network.

17. A system as defined in claim 16 wherein soil gas data representing concentration of said soil gas at a soil site is generated by a method for taking measurements of a soil gas in situ wherein soil gas data representing concentration of said soil gas at a soil site is generated by positioning an apparatus, with a hollow cylindrical body and permeable chamber, below the surface of a soil site, allowing a soil gas to contact an NDIR sensor through a diffusion of said soil gas, measuring and recording data of a concentration of said soil gas diffused within said permeable chamber, and logging said data in a system.

18. A system as defined in claim 17 wherein soil gas data representing concentration of said soil gas at a soil site is generated by a method for taking measurements of a soil gas in situ wherein an apparatus is positioned below the surface of said soil site by supplying power to the apparatus to cause an attached drill bit to rotate from the surface to below the surface of said soil site.

19. A system as defined in claim 16 wherein soil gas data representing concentration of said soil gas at a soil site is sent to a wireless communications network.

20. A system as defined in claim 16 to communicate with one or more remote systems selected from the group consisting of the internet, mobile phone systems, network computers, tablet computers, laptop computers, desktop computers and PDAs.
